(19) United States
(12) Patent Application Publication
(10) Pub. No.: US 2009/0029932 A1
(43) Pub. Date: Jan. 29, 2009

(54) Identification and use of miRNAs for differentiating myeloid leukemia cells

(75) Inventors: Olivier Voinnet, Strasbourg (FR); Charles-Henri Lecellier, Strasbourg (FR); Anne Saumet, Strasbourg (FR); Michel Lanotte, Paris (FR)

Correspondence Address: HARNESS, DICKEY & PIERCE, P.L.C., P.O. BOX 828, BLOOMFIELD HILLS, MI, 48303, US

(73) Assignee: Centre National De La Recherche Scientifique (CNRS), Paris (FR)

(21) Appl. No.: 11/800,044

(22) Filed: May 3, 2007

Related U.S. Application Data
(63) Continuation of application No. PCT/FR2005/002732, filed on Nov. 3, 2005.

(30) Foreign Application Priority Data
Nov. 3, 2004 (FR) 04/11725

(51) Int. Cl. A61K 31/7052; C12Q 1/68; A61P 35/02

(52) U.S. Cl. 514/44; 435/6

(57) ABSTRACT

The invention relates to the use of nucleic acid miRNA derived molecules for producing a drug for treating a myelogenous leukemia and to a method for identifying therapeutic agents or the efficient combination thereof for inducing the differentiation of myelogenous leukemia cells.

### CROSS-REFERENCE TO RELATED APPLICATIONS

This application is a continuation of PCT/FR2005/002732, filed Nov. 3, 2005, which claims priority to French Application No. 04/11725, filed Nov. 3, 2004. Both of these applications are incorporated by reference herein.

### BACKGROUND AND SUMMARY

The present invention concerns a method to identify therapeutic agents for the treatment of myeloid leukemia, a method to identify the miRNAs involved in the differentiation of myeloid leukemia cells and the use of miRNAs or complementary sequences of miRNAs to manufacture a drug intended for the treatment of myeloid leukemia.

The term “RNA silencing” concerns the mechanisms of repression of the expression of a gene mediated by an RNA and using specific sequence interactions. In plants and animals, there are two distinct means of post-transcriptional regulation of the genetic expression that use two different types of small DsRNA.

On the one hand, we have the siDsRNA (short interfering RNA) that are small double strand RNAs (dsRNA) with a length of 21 to 26 nucleotides (nt) and that act like specific sequence mediators for the degradation of mRNA during the mechanism of RNA interference (RNAi). In the small fruit fly, these siRNAs are derived from dsRNA by the action of an enzyme of the RNAse III type called DICER. The siRNAs formed will then associate with the RISC protein complex (RNA-Induced Silencing Complex) that has an endonuclease action. The RISC/siRNA complex formed will then be able to specifically cut the cytoplasmic RNA molecules that present a sequence identical with the siRNA present in the complex. In plants and animals, this mechanism plays an important role of defense. This mechanism, by repressing the proliferation of transposable elements, is also involved in the maintenance of the integrity of the genome.

On the other hand, we also have the miRNAs (micro DsRNA) that are small single strand DsRNA exceedingly preserved during the evolution and whose length is about 20 nucleotides. The miRNAs are generated like the siRNAs, that is, from a double stand precursor matured by the DICER enzyme. A same RNA precursor codes for several miRNAs. Thereby, the miRNAs miR-19b, miR-92, miR-17, miR-18, miR-19a, miR-19b, miR-20 and miR-91 are coded by the same RNA precursor. In addition, the miRNAs miR-23 miR-24 and miR-27 are also coded by a same RNA precursor. Nevertheless, the miRNAs present a certain number of differences with the siRNAs. Thereby, the miRNAs are single strand molecules while the siRNAs are double strand molecules. Although the miRNA miR-196 is able to split mRNA Hox8 (YEKTA et al., Science, vol. 304, p: 594-596, 2004; MANSFIELD, Nat. Genet., vol. 36(10), p: 1079-83, 2004), the majority of animal miRNAs do not induce the endonucleolytic split of targeted RNA. On the contrary, animal miRNAs in general inhibit the translation of targeted RNA by hybridizing with their 3′UTR sequence (untranslated region) via a mechanism that is still not understood (for a review, refer to BARTEL D. P., Cell, vol. 116, p: 281-297, 2004). For all that, animal miRNAs, as opposed to plant miRNAs, present a partial sequence homology with their targets that may justify differences in their mode of action. Finally, as opposed to the siRNAs, the miRNAs do not seem to be involved in the defense mechanisms but rather in development, and especially differentiation. Indeed, the expression of a specific miRNA (miR-181) in haematopoietic stem cells in culture and in vivo increases the fraction of lymphocyte B, suggesting the involvement of this miRNA in the differentiation of haematopoietic cells lymphocyte B (CHEN et al., Science, vol. 303 (5654p, p: 83-6). An indirect indication of the importance of the miRNAs in the process of development in the animal is provided by a suspension of embryogenesis, related to early defects in the differentiation process, in mice whose gene coding for DICER has been mutated (BERSTEIN et al., Nat. Genet., vol. 35, p: 215-7, 2003). Based on these different observations, a model of the mechanism of development has been proposed in which, for each specific cell type, and at a determined stage of development, a set of specific miRNAs influence the expression of a determined fraction of the transcriptome (BARTEL D. P., 2004, previously cited).

Due to the link between the expression of miRNAs and the differentiation process, the expression profile of miRNAs during carcinogenesis is currently raising increasing interest. It has thereby been demonstrated that miRNA let-7 is under-expressed in human lung cancers and its over-expression in a cell line of lung adenocarcinoma inhibits in vitro cell growth (TAKAMIZAWA et al., Cancer Res., vol. 64, p: 3753-3756, 2004).

Leukaemia is qualified as blood cancer and is characterised by the proliferation of leukocytes. Leukaemia may be acute and lead to the death of the patient within weeks or months. This disease may evolve in a lymphocytic form or in a myelogenous form depending on the origin of the cells. The lymphocytic form results from a hyper-proliferation of the progenitors involved in the lymphoid differentiation, while the myelogenous form results from a hyper-proliferation of the progenitors involved in myelogenous differentiation. More specifically concerning the myeloid leukemias, they are treated by a combination of different pharmacological agents that enable the differentiation and consecutive apoptosis of the cancer cells. However, resistance to the treatment often arises, thereby reducing the chances of the patient being cured.

Type 3 acute myeloid leukemia (AML3) or acute promyeloid leukemia (APL) accounts for almost 10% of all cases of acute myeloid leukemia. The cancer cells derived from AML3 are characterised by a blocking of the granulopoiesis (differentiation of the granulocytes) at the promyelocyte phase (DE THE and CHELBI-ALIX, oncogene, vol. 20, p: 7136-9, 2001). The cells blocked at an early stage of differentiation continue to proliferate and accumulate in the bone marrow. Sometimes, this accumulation of cells extends to the peripheral blood circulation, most often provoking the death of the patients by disseminated intra-vascular coagulation. On the molecular level, chromosomic translocation t(15;17) is specifically associated with this type of leukemia and leads to the synthesis of a fusion protein between the retinoic acid receptor a (RARa) and the PML protein. This fusion protein, called PML-RARa interferes in a negative manner with RARa. This interference induces the blocking of the differentiation of the cells at the promyelocyte stage. The clinical treatment of this leukemia uses agents inducing cell differentiation (BENOIT et al., Oncogene, vol. 20, p: 7161-7177, 2001). One of the anti-cancer therapeutic agents most often used in the treatment of AML3 is all trans retinoic acid or ATRA. ATRA allows for the remission of the disease by restoring the differentiation of the leukemia cells and consecutively inducing their death by apoptosis. However, like with other myeloid leukemias, resistance phenomena have arisen demonstrating the limits of the use of ATRA alone in anti-cancer therapy. Different combinations are currently being studied in order to develop more effective protocols. As a result, there is an urgent need to identify new molecules with a therapeutic effect on the myeloid leukemias, new and effective treatment protocols and to also assess the efficiency of a treatment in a patient suffering from myeloid leukemia.

Unexpectedly, the inventors were able to demonstrate that the differentiation of cancer cells derived from a myeloid leukemia is accompanied by a change in the expression of miRNAs, and in particular that the differentiation of cancer cells derived from type 3 acute myeloid leukemia (AML3) is accompanied by a change in the expression of miRNAs miR23a (SEQ ID NO:9, AUCACAUUGCCAGGGAUUUCCA), miR27a (SEQ ID NO:11, UUCACAGUGGCUAAGUUCCGC), and miR24-2 (SEQ ID NO:12 TGGCTCAGTTCAGCAGGAAC) coded by a same RNA precursor (see FIG. 1) of sequence SEQ ID NO:13 (FIG. 2). In view of the involvement of miRNAs in the differentiation process during embryogenesis, the correlation between the expression of miRNAs and the differentiation of cells derived from myeloid leukemia indicates that the miRNAs are also involved in the mechanism of differentiation of the cells derived from myeloid leukemia. The inventors were able to confirm this involvement of miRNAs in the mechanisms of differentiation of cells derived from myeloid leukemia and demonstrate the inhibition of this differentiation in response to an over-expression of the RNA precursor of sequence SEQ ID NO:13.

As a result, the present invention concerns an in vitro method to identify effective therapeutic agents or combinations of therapeutic agents to induce the differentiation of myeloid leukemia cells, characterised in that it comprises the following stages:

i) culture of cells derived from myeloid leukemia,
ii) addition of at least one compound to the culture medium of said cell line,
iii) analysis of the evolution of the level of expression of at least one miRNA coded by the RNA precursor of sequence SEQ ID NO:13 between stages (i) and (ii),
iv) identification of the compounds or combinations of compounds inducing a change in the level of expression of said miRNA between stages (i) and (ii).
Stage (i) of the culture of cells derived from myeloid leukemia may be carried out according to the techniques well known to one skilled in the art. Culture protocols that may be used in the method according to the invention are described, in particular, in BENOIT et al. (2001, previously cited).

According to one preferred embodiment, the method according to the invention allows for the identification of the effective therapeutic agents or combinations of therapeutic agents to treat myeloid leukemia associated with a blocking of granulopoiesis, and particularly to treat a myeloid leukemia associated with a blocking at the promyelocyte stage such as AML3. The cells used in the method of the invention may thereby be derived from an acute myeloid leukemia associated with a blocking of granulopoiesis, and particularly from a myeloid leukemia associated with a blocking of cells at the promyelocyte stage such as AML3.

Advantageously, the cells used may be cells from the cell line NB4 or a cell line derived from the latter, such a derived line may be chosen from among cell lines NB4-LR1 and NB4-LR2 (RUCHAUD et al., 1994, above). A protocol for the culture of cell line NB4 or its derived lines is described in BENOIT et al. (2001, previously cited). The human promyelocytic line NB4 was isolated from a bone marrow sample from a patient with acute promyeloid leukemia (BENOIT et al., 2001, previously cited). The cells bear the translocation t(15;17) and have the ability to differentiate into neutrophile granulocytes under the effect of ATRA.

Lines NB4-LR1 and NB4-LR2, derived from line NB4, present a resistance to the differentiation induced by ATRA. For line NB4-LR1, if the transcriptional response to ATRA is maintained, their differentiation requires an ATRA/cAMP co-treatment. Study of this resistance mechanism was used to identify an alteration in the means of membrane signalling in this line that leads to a blocking of the process of maturation normally triggered by ATRA. For cell line NB4-LR2, its cells express a protein PML-RARa that is truncated in its RARa part. This mutation, located in the domain of the retinoic acid bond of PML-RARa renders these cells insensitive to ATRA and to an ATRA/cAMP mixture. The restoration of the differentiation requires the cooperation between the signalling pathways of the rexinoids, such as SR11237 or BMS 749 (strict RXR agonists (nuclear retinoid X receptor)), and cAMP. Since RARa is no longer functional, it is also possible to use 9-cis-retinoic acid, an agonist of both RAR and RXR, to induce an RXR dependent differentiation.

Advantageously still, the cells put in culture in stage i) may be derived from a sample, in particular a blood sample, from a person suffering from myeloid leukemia. Protocols for the culture of such cells are well known to the professional and are described, in particular, in LANOTTE et al. (Blood., vol. 77, p: 1080-1086, 1991).

According to one preferred embodiment, the compound(s) used in stage (ii) of the method according to the invention may be of any type, in particular protein, carbohydrate or lipid. The professional may thereby easily and quickly test compounds in the method according to the invention that he considers may have an effect on the differentiation of cells derived from a myeloid leukemia.

According to another preferred embodiment, the compound(s) used in stage (ii) of the method according to the invention may be therapeutic agents used in the treatment of other diseases, and more particularly in the treatment of other cancers. One skilled in the art may thereby easily and quickly test therapeutic agents known in the treatment of other diseases in the method according to the invention, that he considers may have an effect on the differentiation of cells derived from a myeloid leukemia.

According to another preferred embodiment of the invention, the compound(s) used in stage (ii) of the method according to the invention may be therapeutic agents used in the treatment of myeloid leukemia. The method according to the invention may then be used to determine the optimum doses and/or combinations of therapeutic agents to obtain a differentiation of the cells. By way of example of such therapeutic agents, we can in particular mention cAMP, arsenic, the interferons, TNF, retinoic acid and retinoid derivatives such as ATRA, and the rexinoids. The compound added to stage (ii) of the method according to the invention may be directly added to the cell culture medium at a concentration ranging from 1 pM to 1 M, preferably between 1 nM and 100 mM, and in a particularly preferred manner between 100 nM and 1 mM.

Stage (iii) may be carried out according to the analysis techniques familiar to one skilled in the art. For example, this stage may use the northern blot technique, protection with Rnase, quantitative RT-PCR or even use DNA chips integrating oligonucleotides complementary to miRNAs. Preferably, this stage of analysis may use the northern blot technique according to the protocol described in LLAVE et al. (Plant Cell, vol. 14, p: 1605-1619, 2002). To use such an analysis, the RNA of the cells put in culture in stage (i), before and after the addition of a therapeutic agent in stage (ii), may be extracted according to extraction techniques known to one skilled in the art. In particular, cell samples may be taken on a daily basis. The purified RNA may then be deposited on an electrophoresis gel. After migration of the electrophoresis gel and transfer of RNA on membrane, the membrane may be hybridised with a labelled, cold (biotine, etc.) or radioactive (P³², P³³, etc.) probe, presenting a fully or partly complementary sequence with the RNA precursor of sequence SEQ ID NO:13, or at least one miRNA coded by this precursor. The length of the sequence of the probe is greater or equal to 10 nucleotides, preferably 15 nucleotides, and in an especially preferred manner 20 nucleotides. The sequence of the probe is fully or partly complementary to the sequence of the precursor RNA SEQ ID NO:13, preferably to the sequence of at least one miRNA coded by this precursor, and in an especially preferred manner to at least one miRNA chosen from among miR23a (SEQ ID NO:9), miR27a (SEQ ID NO:11), and miR24-2 (SEQ ID NO:12). After hybridisation and washing of the membrane, the hybridisation signal corresponding to the miRNA analysed may be quantified according to the techniques well known to the professional, in particular by using a Phosphoimager®. The hybridisation signal obtained with a probe complementary to this miRNA may then be standardised with the hybridisation signal obtained with a probe complementary to a transcript constitutively expressed in the cells, such as RNA 28S. The standardised value obtained for each sample corresponds to the level of expression of the miRNA in the cells for each condition tested.

According to one specific mode of application of the method according to the invention, the cells used in stage (i) may first be transfected using techniques known to one skilled in the art by a construction containing a reporter gene, such as the gene of GFP, and potentially a resistance gene, such as a resistance gene to hygromycine or neomycine. In addition, the reporter gene contains at least one sequence fully or partly complementary to the precursor RNA sequence SEQ ID NO:13, preferably to at least one sequence of a miRNA coded by this precursor, and in an especially preferred manner, to at least one miRNA chosen from among miR23a (SEQ ID NO:9), miR27a (SEQ ID NO:11), and miR24-2 (SEQ ID NO:12). Advantageously, the length of said complementary sequences ranges from 10 to 100 nucleotides, preferably between 15 and 50 nucleotides, and in an especially preferred manner between 18 and 25 nucleotides. The protocols that can be used to obtain such a construction are known to one skilled in the art. Such a protocol is in particular described for siRNAs in MANSFIELD et al. (2004, previously cited). The use of such a construction helps considerably simplify the analysis of the level of expression of different miRNA since it does not require the extraction of RNA. In fact, an animal miRNA has been shown to be able to induce the split of an RNA when the latter presents a sequence that is perfectly complementary to the miRNA. The expression of the reporter gene, in particular GFP, then depends on the expression of miRNA of which a complementary sequence is present within the sequence coding for the reporter gene. Thereby, according to whether the therapeutic agent reduces or increases the RNA precursor or at least one miRNA coded by it, we will observe an increase or a decrease in the expression of the reporter gene respectively. The expression of the reporter gene may be monitored using the techniques familiar to one skilled in the art and, in particular, in the case of GFP, by monitoring the emission of fluorescence of transfected cells.

Stage (iv) consists of the identification of compounds or combinations of compounds inducing an increase and/or decrease in the level of expression of the precursor RNA SEQ ID NO:13, or at least one miRNA coded by this precursor, preferably one miRNA chosen from among miR23a (SEQ ID NO:9), miR27a (SEQ ID NO:11), and miR24-2 (SEQ ID NO:12). According to a specific embodiment of the invention, stage (iv) consists of the identification of compounds or combinations of compounds inducing a reduction in the level of expression of at least one of said miRNAs. In this embodiment, the reduction in the level of expression of at least one of said miRNAs may appear between the day following the addition of the therapeutic agent (D1) and the fourth day of treatment (D4).

A second object of the present invention concerns an in vitro method to identify miRNAs associated with the differentiation of cells derived from a myeloid leukemia, characterised in that it comprises the following stages:

i) culture of a cell line derived from a myeloid leukemia,
ii) addition, in the culture medium, of at least one compound inducing the differentiation of said cell line,
iii) analysis of the evolution of the level of expression of at least one miRNA, or a precursor of miRNAs between stages (i) and (ii),
iv) identification of the miRNAs that present a variation in their expression profile during the differentiation.
The culture stage (i) of cells derived from a myeloid leukemia may be carried out as described above.

According to one preferred means of achievement, the method according to the invention can be used to identify miRNAs whose expression is associated with the differentiation of cells derived from a myeloid leukemia associated with a blocking of the granulopoiesis of said cells, preferably associated with a blocking at the promyelocytic stage such as AML3. Advantageously, the cell line derived from a myeloid leukemia associated with a blocking of granulopoiesis may be the cell line NB4 or lines derived from it, in particular lines NB4-LR1 and NB4-LR2 described above. The culture of said cell lines may be carried out as described in BENOIT et al. (2001, previously cited).

Stage (ii) for the addition of compounds inducing the differentiation may use therapeutic agents that are currently used in the treatment of cancer, and preferably in the treatment of myeloid leukemia. By way of example of therapeutic agents that can be used in stage (ii) of the method according to the invention, in particular cAMP, arsenic, the interferons, TNF, retinoic acid and the retinoid derivatives, such as ATRA, the rexinoids can be mentioned. The therapeutic agent used may be directly added to the cell culture medium at a concentration ranging from 1 pM to 1 M, preferably between 1 nM and 100 mM, and in an especially preferred manner between 100 nM and 1 mM.

According to one preferred embodiment of the method according to the invention, the compounds inducing the differentiation for the line NB4 may be chosen from among ATRA and an ATRA/cAMP mixture. To obtain a differentiation of said cells, the concentration of ATRA used should range from 1 nM to 1 mM, preferably between 10 nM and 100 μM, in an especially preferred manner between 100 nM and 10 μM. The ATRA may also be used in combination with cAMP present at a concentration ranging from 100 nM to 100 mM, preferably between 1 μM and 10 mM, in an especially preferred manner between 10 μM and 1 mM.

According to a second preferred embodiment of the invention, a compound inducing the differentiation for the line NB4-LR1 may be an ATRA/cAMP mixture. The preferred concentrations for these therapeutic agents are the same as those described above. According to a third preferred means of achievement of the invention, a compound inducing the differentiation for the line NB4-LR2 may be an cAMP/rexinoids mixture, such as an cAMP/SR11237 or cAMP/BMS 749 mixture, or an cAMP/9-cis-retinoic acid mixture. To obtain a differentiation of said cells, the concentration in rexinoids, such as SR11237 or cAMP/BMS 749, or 9-cis-retinoic acid may range from 1 nM to 1 mM, preferably between 10 nM and 100 μM, in an especially preferred manner between 100 nM and 10 μM. The preferred concentrations for the cAMP are the same as those described above.

Stage (iii) of analysis may be carried out as described above, but by using sequences complementary to the sequence of at least one miRNA as a probe. Sequence of miRNAs are in particular described in application WO 03/029459 or on the World Wide Web internet site at sanger.ac.uk/Software/Rfam/mirna/index.shtml. By way of an internal control, we can use a probe fully or partly complementary to the precursor RNA sequence SEQ ID NO:7 (see FIG. 3), preferably at least one miRNA coded by this precursor, and in an especially preferred manner to at least one miRNA chosen from among miR-17 (SEQ ID NO:1), miR-18 (SEQ ID NO:2), miR-19a (SEQ ID NO:3), miR-19b (SEQ ID NO:4), miR-20 (SEQ ID NO:5), miR-91 (SEQ ID NO:8) and miR-92 (SEQ ID NO:6).

Stage (iv) consists of the identification of miRNAs that present a variation in their expression profile during the differentiation of the cell line used. According to one preferred embodiment, stage (iv) consists of the identification of miRNAs with an expression profile that is identical or similar to at least one miRNA coded by the precursor RNA SEQ ID NO:7, preferably at least one miRNA chosen from among miR-17 (SEQ ID NO:1), miR-18 (SEQ ID NO:2), miR-19a (SEQ ID NO:3), miR-19b (SEQ ID NO:4), miR-20 (SEQ ID NO:5), miR-91 (SEQ ID NO:8) and miR-92 (SEQ ID NO:6).

miRNA presenting an expression profile identical to that of at least one miRNA coded by the precursor RNA SEQ ID NO:7, indicates a miRNA whose variations in the level of expression follow the same kinetics and the have the same amplitude as those of at least one miRNA coded by the precursor RNA SEQ ID NO:7 during the differentiation of the cells of the cell line used, and in particular the cells of cell line NB4 or a cell line derived from it. miRNA presenting an expression profile similar to that of at least one miRNA coded by the precursor RNA SEQ ID NO: 7, indicates a miRNA whose variations at the level of expression follow a kinetics with a shift of several days, typically one or two days, and/or has an amplitude greater or lower than the variations in the level of expression of at least one miRNA coded by the precursor RNA SEQ ID NO:7 during the differentiation of the cells from cell line NB4 or a derived cell line.

According to one preferred embodiment of the invention, the miRNAs identified present an increase in their level of expression in response to the addition of a therapeutic agent inducing the differentiation, such as ATRA or an ATRA/cAMP mixture, between the day of treatment (D0) and day four of treatment (D4), preferably between the first and third day of treatment with said therapeutic agent. According to a second preferred embodiment of the invention, the miRNAs identified present a reduction in their level of expression in response to the addition of a therapeutic agent inducing the differentiation, such as ATRA, between the second (D2) and the fourth day of treatment (D4) with said therapeutic agent. A third object of the present invention concerns the use, to manufacture a drug for the treatment of myeloid leukemia, from a nucleic acid molecule chosen from among the precursor RNA miR23a/24-2 (SEQ ID NO:13), a sequence derived from such an RNA, a complementary sequence from such RNA and a sequence derived from such a complementary sequence. Advantageously, said drug is a nucleic acid molecule chosen from among a complementary sequence of the precursor RNA miR23a/24-2 (SEQ ID NO:13) and a sequence derived from such a complementary sequence.

According to another preferred embodiment of the invention, the invention comprises the use, to manufacture a drug for the treatment of myeloid leukemia, of at least one nucleic acid molecule presenting a sequence chosen from among:

i) the sequence of miR23a (SEQ ID NO:9), a sequence derived from miR23a, the complementary sequence of miR23a, a sequence derived from such a complementary sequence,
ii) the sequence of miR27a (SEQ ID NO:11), a sequence derived from miR27a, the complementary sequence of miR27a, a sequence derived from such a complementary sequence,
iii) the sequence of miR24-2 (SEQ ID NO:12), a sequence derived from miR24-2, the complementary sequence of miR24-2 and a sequence derived from such a complementary sequence.

Advantageously, said drug comprises a nucleic acid molecule chosen from among a complementary sequence of miR23a (SEQ ID NO:9), miR27a (SEQ ID NO:11) and miR24-2 (SEQ ID NO:12), and the sequences derived from such complementary sequences.

Preferably, the purpose of the invention includes the use of at least one of said nucleic acid molecules, to manufacture a drug for the treatment of a myeloid leukemia associated with a granulopoiesis blocking, and in an especially preferred manner to a blocking of the promyelocytic stage, such as AML3. The nucleic acid molecules may be used in single strand or double strand form, preferably in single stand form. The nucleic acids may be selected among DNA, RNA or the modified nucleic acids such as the ribonucleotides or the deoxyribonucleotides presenting a sugar group or a modified carbon group. The RNA or DNA molecules used in the present invention may also contain one or several modified nucleotides, that is a ribonucleotide or natural deoxyribonucleotide substituted by a synthetic analogue of a nucleotide. Such analogues of nucleotides may, for example, be located at the 3′ or 5′ end of the nucleic acid molecule.

Preferred synthetic analogues of nucleotides are selected from among the ribonucleotides presenting a sugar group or a modified carbon group. Preferably, the ribonucleotides presenting a modified sugar group present a 2′-OH group replaced by a group selected from among a hydrogen atom, a halogen, an OR, R, SH, SR, NH₂, NHR, NR₂ or CN group, where R is an alkyl, alkenyl or alkynyl group of 1 to 6 carbon and the halogen is fluorine, chlorine, bromine or iodine. Preferably, the ribonucleotides presenting a modified carbon group have their phosphoester group bound to the adjacent ribonucleotide that is replaced by a modified group such as a phosphtioate group. In addition, it is also possible to use ribonucleotides presenting a purine or modified pyrimidine core. As examples of such modified cores, we can in particular mention the uridines or cytidines modified in position 5, such as 5-(2-amino)propyl uridine and 5-bromouridine, the adenosines and guanosines modified in position 8, such as 8-bromoguanosine, the denitrogenous nucleotides, such as 7-deaza-adenosine, the N- and O-alkylated nucleotides, such as N6-methyl-adenosine. These different modifications may also be combined.

The nucleic acid molecules used in the present invention may be obtained by the methods of chemical synthesis or by the methods of molecular biology, in particular by transcription from DNA matrixes or plasmids isolated from recombinant micro-organisms. Preferably, this stage of transcription uses phage polymerase RNA such as polymerase RNA T7, T3 or SP6.

Derived sequence refers to a sequence presenting an identity of at least 80%, preferably at least 90%, and in an especially preferred manner at least 95% with a reference sequence. The determination of a sequence identity is carried out according to the following formula:

I=n/L

where I represents the percentage identity (%), n is the number of identical nucleotides between a given sequence and a given sequence of miRNA and L is the length of the sequence. Nucleotides A, C, G and U may correspond to ribonucleotides, deoxyribonucleotides and/or analogues of nucleotides, such as synthetic nucleotide analogues. In addition, the nucleotides may be substituted by nucleotides forming analogue hydrogen bonds with a complementary nucleic sequence. Thereby, the nucleotide U may be substituted by a nucleotide T.

The length of the nucleic acid molecules used to manufacture a drug for the treatment of myeloid leukemia preferably ranges from 15 to 100 nucleotides, preferentially between 18 and 80 nucleotides and in an especially preferred manner between 18 and 30 nucleotides. The length of the mature miRNA molecules ranges from 19 to 24 nucleotides, and more particularly from 21, 22 or 23 nucleotides. Advantageously, the length of the complementary sequence to a miRNA ranges from 19 to 24 nucleotides. However, it is possible to use the sequence derived from a precursor of miRNAs with a length ranging from 50 to 90 nucleotides, most often between 60 and 80 nucleotides, but that may also have a length exceeding 100 nucleotides. The nucleic acid molecules may be administered by methods of gene transfer familiar to one skilled in the art.

Common methods of gene transfer include calcium phosphate, DEAE-Dextran, electroporation, microinjection, viral methods and the cationic liposomes (GRAHAM and VAN DER EB, Virol., vol. 52, p: 456, 1973; McCUTHAN and PAGANO, J. Natl. Cancer Inst., vol. 41, p: 351, 1968; CHU et al., Nucl. Acids Res., vol. 15, p: 1311; FRALEY et al., J. Biol. Chem., vol. 255, p: 10431, 1980; CAPECCHI et al., Cell, vol. 22, p: 479, 1980; FELGNER et al., Proc. Natl. Acad. Sci. USA, vol. 84, p: 7413, 1987).

The nucleic acid molecules to administer may be in solution form, in particular that of an injectable solution, a cream, a tablet or even a suspension. The vehicle may be any pharmaceutical vehicle. Preferably, a vehicle able to improve the admission of the nucleic acid molecules in the cells will be used. Such vehicles include, in particular, the liposomes, preferably the cationic liposomes.

An effective quantity of nucleic acid molecules to administer to a patient may be easily determined by the professional. By way of example, an effective quantity of nucleic acid molecules ranges from 0.001 mg to 10 g/kg of patient to treat, preferably from 0.01 mg to 1 g/kg, and in an especially preferred manner from 0.1 to 100 mg/kg.

### BRIEF DESCRIPTION OF THE DRAWINGS

FIG. 1 shows Organization of miRNAs on Chromosomes 13 and 19;

FIG. 2 shows the sequence (SEQ ID NO:13) of an RNA precursor encoding miRNAs miR-23a, miR-27a, and miR-24-2;

FIG. 3 shows the sequence (SEQ ID NO:7) of an RNA precursor coding for the miRNAs miR-17 to miR-92;

FIG. 4 shows an example of the coloration of NB4 to NBT cells before and after three days of treatment with ATRA;

FIG. 5 shows an expression profile of the different miRNAs analyzed in the ATRA treated NB4 cells;

FIG. 6 shows different expression profiles of miR-23a in NB4-LR1 and NB4-LR2 cells treated with ATRA;

FIG. 7 shows the sequence (SEQ ID NO:16) of an RNA precursor coding for miRNAs miR-15a and miR-16;

FIG. 8 shows the sequence (SEQ ID NO:10) of DNA corresponding to the RNA precursor (SEQ ID NO:7);

FIG. 9 shows effects on NB4 cell differentiation of expression of MSCV-IRES-eGFP (MIE) vector-delivered miRNA constructs during ATRA treatment;

FIG. 10 shows the percent of NBT-positive cells obtained by expression of the MIE vector-delivered constructs;

FIG. 11 shows results that the cells infected by the MIE-miRNAs vectors present a level of expression for the miRNAs coded by the MIE-miRNAs vector used to infect them;

FIG. 12 shows results that only the vector integrating the whole miR23a/24-2 precursor is able to block the differentiation of the NB4 cells in the presence of ATRA;

FIG. 13 shows Northern blot experiments verifying over-expression of miRNAs;

FIG. 14 shows results that the complementation in trans of the different miRNAs of the miR23a/24-a precursors do not block the differentiation of the NB4 cells in the presence of ATRA; and

FIG. 15 shows Northern blot experiments verifying over-expression of miRNAs.

### DETAILED DESCRIPTION

The following examples are provided by way of illustration and do not limit the extent of the present invention.

### Example 1

### Differentiation of NB4 and NB4-LR1-Cells in the Presence of ATRA and/or cAMP

The cells from cell line NB4 and cell line NB4-LR1, resistant at maturation only by ATRA, were grown as described in LANOTTE et al. (1991, previously cited) and in RUCHAUD et al. (Proc. Natl. Acad. Sci., vol. 91, p: 8428-8432, 1994). The cells were then treated for 4 days in the presence of 1 μM of all-trans retinoic acid (ATRA, SIGMA-ALRICH) alone or with the addition of 100 μM of an cAMP analogue (8-CPT-cAMP, SIGMA-ALRICH). The cell proliferation was determined on a daily basis by counting the cells using a cell counter (BECKMAN COULTER FRANCE SA) from day 0, following the addition of the therapeutic agent, to day 4. The results show that the different treatments induce a reduction in the proliferation.

In parallel, the evolution of the differentiation was determined on a daily basis throughout the treatment. The granulocytic differentiation was simultaneously evaluated according to the morphological and biochemical criteria. The morphological analysis was carried out after May-Grünwald colouring. The biochemical analysis was carried out by a coloration test based on the reduction of NBT (nitro blue tetrazolium) that is used to measure the oxidative ability of the mature cells to reduce the NBT colorant. For this purpose, 0.5 to 1×10⁵ cells were centrifuged for 5 minutes at 190 g. The cell sediment was then recovered in 200 μl of saline phosphate buffer (PBS) with the addition of NBT (SIGMA ALDRICH, 1 mg/ml) and PMA (Phorbol 12-myristate 13-acetate, SIGMA, 10⁻⁷M) and then was incubated for 20 minutes at 37° C. The cells were then collected on slides by Cytospin® centrifugation, then observed by phase contrast microscopy. A minimum of 200 cells per slide were examined under light microscope and a differentiation percentage was calculated on the basis of the number of positive NBT cells. The results obtained are summed up in table I below:

TABLE I

| | | Evolution of the percentage of | | | | |
|---|---|---|---|---|---|---|
| | | cells differentiated during the | | | | |
| | | treatment (in days) | | | | |
| Cell line | Treatment | 0 | 1 | 2 | 3 | 4 |
| NB4 | ATRA | 0% | 6% | 20% | 43% | 95% |
| | ATRA + cAMP | 0% | 6% | 30% | 80% | 100% |
| NB4-LR1 | ATRA | 0% | 0% | 2% | 5% | 5% |
| | ATRA + cAMP | 0% | 5% | 25% | 75% | 95% |

The results show that the ATRA/cAMP co-treatment enables a differentiation of NB4 and NB4-LR1 cells. However, only the cells in the NB4 cell line differentiate in the presence of ATRA alone. FIG. 4 shows an example of the coloration of NB4 to NBT cells before and after three days of treatment with ATRA. A sharp change in the morphology of the cells is observed that results in their differentiation.

### Example 2

### Expression of miRNAs During the Differentiation of NB4 Cells Induced by ATRA

In a first series of experiments, the expression of different miRNAs was evaluated during the differentiation of NB4 cells in the presence or absence of ATRA. The expression of the following miRNAs was determined in particular:

(SEQ ID NO:9)

| | |
|---|---|
| miR23a | AUCACAUUGCCAGGGAUUUCCA |

(SEQ ID NO:11)

| | |
|---|---|
| miR27a | UUCACAGUGGCUAAGUUCCGC |

(SEQ ID NO:12)

| | |
|---|---|
| miR24-2 | UGGCUCAGUUCAGCAGGAACAG |

(SEQ ID NO:14)

| | |
|---|---|
| miR15a | UAGCAGCACAUAAUGGUUUGUG |

(SEQ ID NO:15)

| | |
|---|---|
| miR16 | UAGCAGCACGUAAAUAUUGGCG |

(SEQ ID NO:4)

| | |
|---|---|
| miR19b | UGUGCAAAUCCAUGCAAAACUGA |

(SEQ ID NO:6)

| | |
|---|---|
| miR92 | UAUUGCACUUGUCCCGGCCUGU |

(SEQ ID NO:3)

| | |
|---|---|
| miR19a | UGUGCAAAUCUAUGCAAAACUGA |

(SEQ ID NO:5)

| | |
|---|---|
| miR20 | UAAAGUGCUUAUAGUGCAGGUA |

(SEQ ID NO:1)

| | |
|---|---|
| miR17 | CAAAGUGCUUACAGUGCAGGUAGU |

(SEQ ID NO:2)

| | |
|---|---|
| miR18 | UAAGGUGCAUCUAGUGCAGAUA |

(SEQ ID NO:8)

| | |
|---|---|
| miR91 | ACUGCAGUGAAGGCACUUGU |

(SEQ ID NO:17)

| | |
|---|---|
| let-7a | UGAGGUAGUAGGUUGUAUAGUU |

(SEQ ID NO:18)

| | |
|---|---|
| let-7d | AGAGGUAGUAGGUUGCAUAGU |

(SEQ ID NO:19)

| | |
|---|---|
| miR15b | UAGCAGCACAUCAUGGUUUACA |

(SEQ ID NO:20)

| | |
|---|---|
| miR142S | CAUAAAGUAGAAAGCACUAC |

(SEQ ID NO:21)

| | |
|---|---|
| miR223 | UGUCAGUUUGUCAAAUACCCC |

(SEQ ID NO:22)

| | |
|---|---|
| miR320 | AAAAGCUGGGUUGAGAGGGCGAA |

(SEQ ID NO:23)

| | |
|---|---|
| miR422b | CUGGACUUGGAGUCAGAAGGCC |

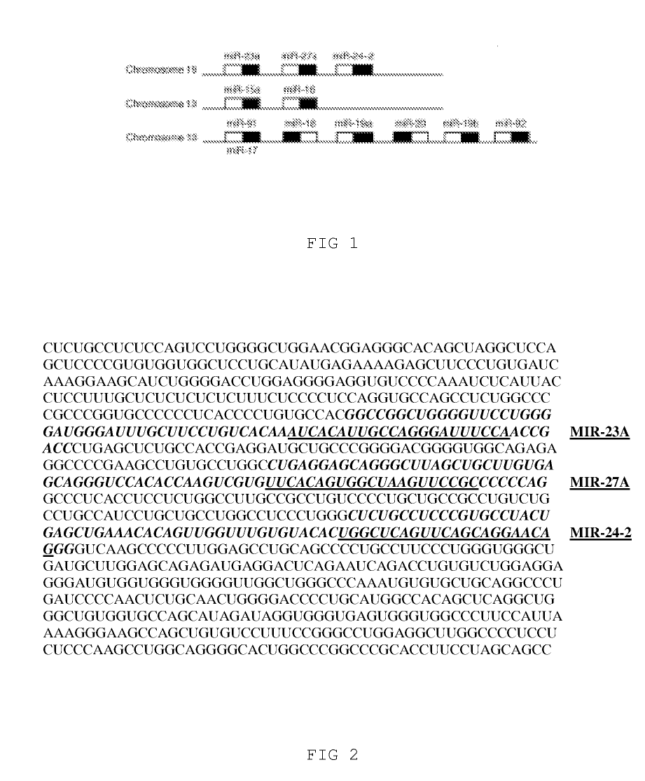

Some of these miRNAs belong to a same precursor, thereby (A) the miRNAs miR-19b, miR-92 miR-17, miR-18, miR-19a, miR-19b, miR-20, miR-91 and miR-92, (B) the miRNAs miR15a and miR16, and (C) the miRNAs miR23a, miR27a and miR24-2 (see FIG. 1). A model submits that such miRNAs generated from a same RNA precursor present a same expression profile (LEE et al., Embo J., vol. 21, p: 4663-4670, 2002).

The total RNA was extracted from the cells of the NB4 cell lines treated or not treated with 1 μM of ATRA, at the same time intervals as in example 1, and using the Tri-Reagent® kit (SIGMA) according to the manufacturer's instructions. The analysis of the low molecular weight RNA by northern blot was carried out as described in LLAVE et al. (2002, previously cited). All of the northern blot experiments were carried out in double. DNA oligonucleotides complementary to the sequences of the miRNAs analysed were labelled at their end with ATP γ-P³² using the polynucleotide kinase T4 (NEW ENGLAND BIOLABS) by following the manufacturer's instructions.

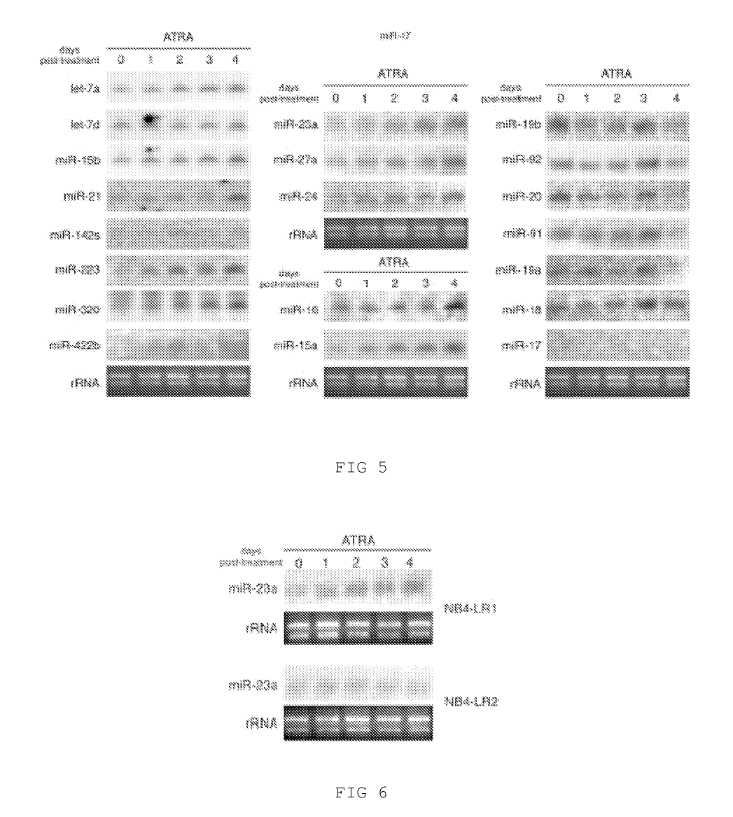

FIG. 5 shows the expression profile of the different miRNAs analysed in the NB4 cells after 0, 1, 2, 3 and 4 days of treatment. The quantity of RNA in each well was controlled by coloration of the gel with ethidium bromide and visualisation of the ribosomal RNA (rRNA) under UV light. The results show that the induction of the differentiation of the NB4 cells in the presence of ATRA induces a modification in the expression of numerous miRNAs (see FIG. 5). In addition, the miRNAs that seem to belong to a same precursor effectively present a similar expression profile over time.

In a more detailed manner, the results obtained demonstrate a modulation in the level of expression of the miRNAs miR-19b, miR-23a and miR-92 during the differentiation of the NB4 cells in response to treatment with ATRA. In the case of miR-23a, its level of expression increases over time in response to the treatment with ATRA.

The expression profile of miR-19b and miR-92 differs from that of miR-23. This expression profile corresponds to a first increase in the level of expression of miR-19b and miR-92, immediately after the treatment (D0) with a maximum of expression on day three of treatment. Finally, the level of expression of miR-19b and miR-92 drops between day 3 and day 4 of treatment (between D3 and D4) while the differentiation of the granulocytes is complete (see table I). In addition, the level of expression of miR-19b and miR-92 on day 4 of treatment is lower than their level of expression in the non treated cells.

### Example 3

### Expression of miRNAs in the Cells of NB4 and NB4-LR1 Cell Lines in Response to a Treatment with ATRA

To formally establish the correlation between the expression of the different miRNAs identified and the differentiation in granulocytes, cells from NB4 and NB4-LR1 cell lines were cultivated in the presence or absence of ATRA as described in example 1. Northern blot experiments were carried out according to the protocol described in example 2. The different northern blot experiments were carried out with probes complementary to the miRNAs miR-23a, miR-17, miR-18, miR-19a, miR-19b, miR-20, miR-23 and miR-92.

The results obtained show that the expression profile of miR-23 is similar in the NB4 and NB4-LR1 cells in response to a treatment with ATRA (see FIGS. 5 and 6). However, an increase in the expression of miR23a is not observed in the case of treatment of NB4-LR2 cells with ATRA. However, the results demonstrated that the different miRNAs miR-17, miR-18, miR-19a, miR19b, miR-20 and miR-92, which are coded by a same RNA precursor, present a different expression profile between the NB4 and NB4-LR1 cells in response to the treatment with ATRA.

### Example 4

### Expression of miRNA in Cells of NB4 and NB4-LR1 Cell Lines in Response to a Simultaneous Treatment with ATRA and cAMP

To confirm the correlation between the expression of different miRNAs, and in particular miR-17, miR-18, miR-19a, miR19b, miR-20 and miR-92 and the differentiation in granulocytes, cells from NB4 and NB4-LR1 cell lines were cultivated in the presence or absence of ATRA/cAMP as described in example 1. Following an ATRA/cAMP co-treatment, the differentiation of the cells in the NB4-LR1 cell line in granulocytes is restored. Northern blot experiments were carried out according to the protocol described in example 2. The different northern blot experiments were carried out with probes complementary to the miRNAs miR-17, miR-18, miR-19a, miR-19b, miR-20 and miR-92.

The results demonstrated that the expression profile of these different miRNAs is identical between the NB4 and NB4-LR1 cells in response to the ATRA/cAMP co-treatment. However, the expression profile of these miRNAs differs from that observed in the NB4 cells in response to the ATRA treatment alone. In response to the ATRA/cAMP co-treatment, the miRNAs miR-17, miR-18, miR-19a, miR-19b, miR-20 and miR-92 demonstrate a maximum expression on day 2 of treatment, followed by a considerable reduction in their level of expression between day 2 and day 3 of treatment. Finally, their initial level of expression is re-established between day 3 and day 4 of expression. The induction and consecutive drop in the level of expression of the miRNAs miR-17, miR-18, miR-19a, miR-19b, miR-20 and miR-92 therefore operates more early in response to the ATRA/cAMP co-treatment (compared with the treatment with only ATRA), just like the differentiation of the NB4 cells in granulocytes (see table 1).

The results obtained thereby support the correlation between the expression of certain miRNAs, and in particular the miRNAs miR-17, miR-18, miR-19a, miR19b, miR-20 and miR-92 and the differentiation of the NB4 and NB4-LR1 cells in granulocytes. In addition, the different expression kinetics from the miRNAs studied between the treatment with ATRA and the ATRA/cAMP co-treatment may be justified by the different differentiation kinetics between the two treatments. Finally, the expression kinetics of the miRNAs miR-17, miR-18, miR-19a, miR19b, miR-20 and miR-92 thereby successively shows an induction of their expression with the initiation of the differentiation, then an inhibition of their expression at the end of differentiation.

### Example 5

### Differentiation of NB4 Cells in Response to an Over-Expression of Different miRNAs

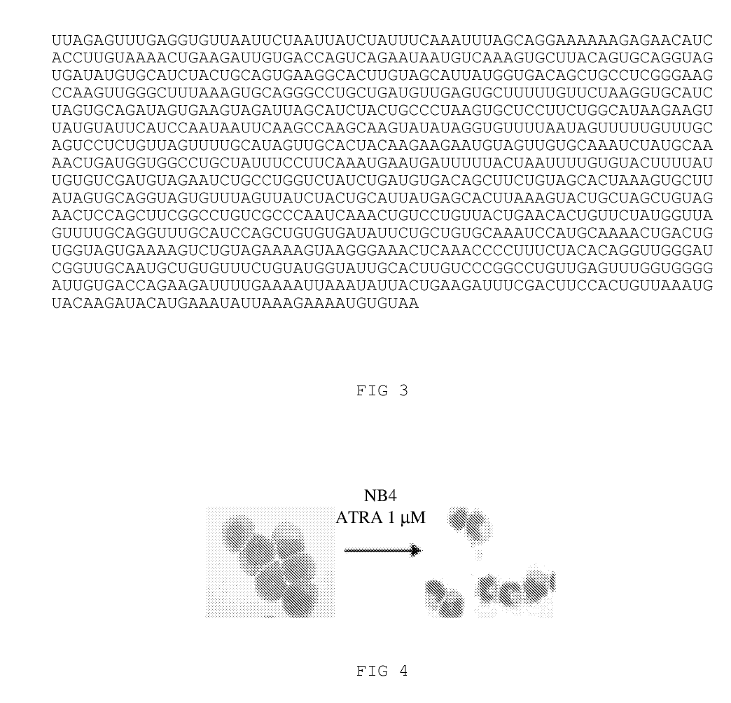

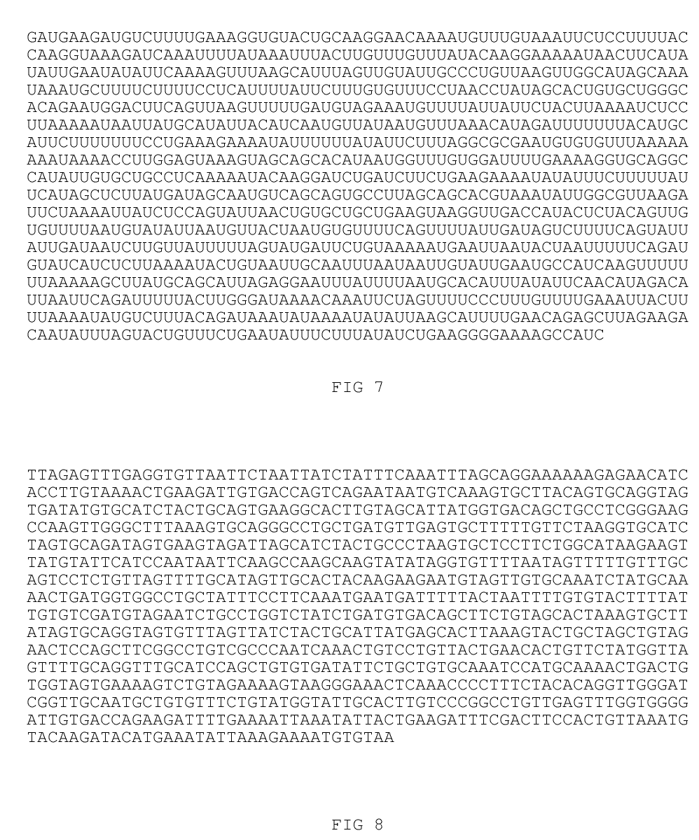

To determine the possible involvement of certain miRNAs analysed in granulopoiesis, the genome sequences coding for the precursor RNA of sequence SEQ ID NO:7 coding for the miRNAs miR17/92 (see FIG. 3, the complementary sequence of the sequence coding for the precursor RNA of sequence SEQ ID NO:7 is represented in FIG. 8, SEQ ID NO:10), for the precursor RNA of sequence SEQ ID NO:13 coding for the miRNAs miR23a/24-2 and for the precursor RNA of sequence SEQ ID NO:16 (see FIG. 7) coding for the miRNAs miR16/15a were cloned up the line from the internal ribosomic entry site (IRES) of the MIE vector (MSCV IRES EGFP (enhanced green fluorescent protein), SYSTEMIX) as described in CHANGCHUN et al. (Blood, vol. 94(2), p: 793-802, 1999). The production of supernatants containing different MIE-miRNA retroviruses (MIE in particular containing the genome sequence SEQ ID NO:10 under control of a pol II promoter) in the Bosc 23 cell line (PEAR et al., Proc. Natl. Acad. Sci. USA, vol. 90, p: 8392-8396, 1993) was then carried out as described in LAVAU et al. (EMBO J., vol. 16, p: 4226-4237, 1997). Cells from the NB4 line were then infected by different retroviruses and selected according to the protocol described in CHANGCHUN et al. (1999, previously cited).

The cells in the NB4 cell line infected by the MIE vector alone or by the different MIE-miRNA vectors, were then cultivated as described in example 1 in the presence or absence of ATRA. The evolution of the differentiation was determined on a daily basis for the different cultures as described in example 1.

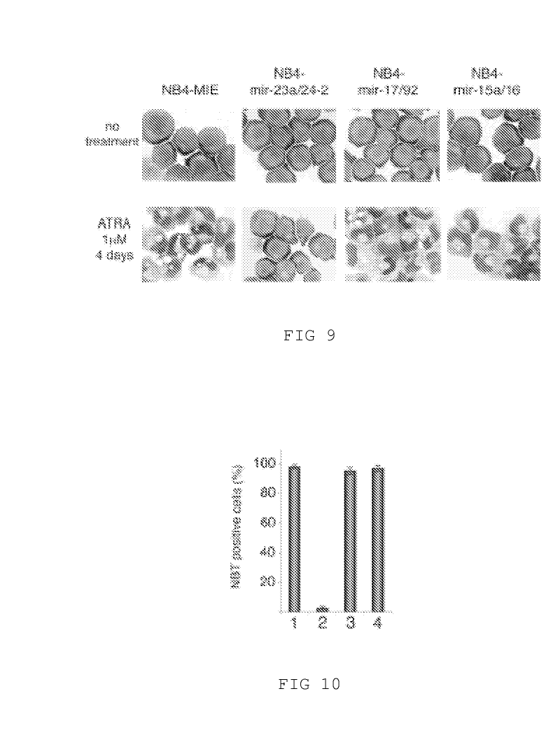

The results show that, as opposed to the cells infected by the MIE vectors alone, MIE-miR17/miR92 and miR15a/16, which differentiate only after four days of culture in the presence of ATRA, the cells infected by the MIE-miR23a/miR24-2 vector do not differentiate in the same conditions (see FIGS. 9 and 10). To confirm the over-expression of the miRNAs in the infected NB4 cells, northern blot experiments were carried out according to the protocol described in example 2. The different northern blot experiments were carried out with probes complementary to the miRNAs miR-23a, miR16 and miR-92.

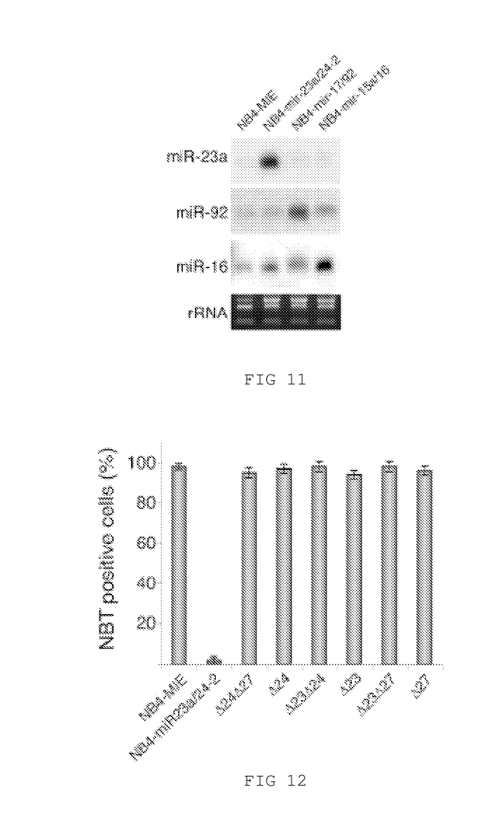

The results confirm that the cells infected by the MIE-miRNAs vectors present a level of expression for the miRNAs coded by the MIE-miRNAs vector used to infect them (see FIG. 11). Therefore, and unexpectedly, the results suggest that the miRNAs miR23a, miR27a and miR24-2 are potentially involved in the differentiation of NB4 cells in the presence of ATRA, and more specifically that these miRNAs “negatively” regulate the differentiation of the granulopoiesis in the NB4 cells.

### Example 6

### Differentiation of NB4 Cells in Response to an Over-Expression of Different miRNAs Coded by the miR23a/24-2 Precursor

To individually determine the involvement of the different miRNAs coded by the miR2a/24-2 precursor in the granulopoiesis induced by ATRA, the genome sequence coding for the precursor RNA of sequence SEQ ID NO:13 coding for the miRNAs miR23a/24-2, as well as the different constructions presenting a deletion for one or two miRNAs coded by the latter (Δ24Δ27, Δ24, Δ23Δ24, Δ23, Δ23Δ27 and Δ27) were cloned up the line from the internal ribosome entry site (IRES) of the MIE vector described above. Cells from the NB4 cell line were then infected by these different retrovirus vectors, selected according to the protocol described in example 5 and then cultivated as described in example 1 in the presence or absence of ATRA. The evolution of the differentiation was determined on a daily basis for the different cultures as described in example 1.

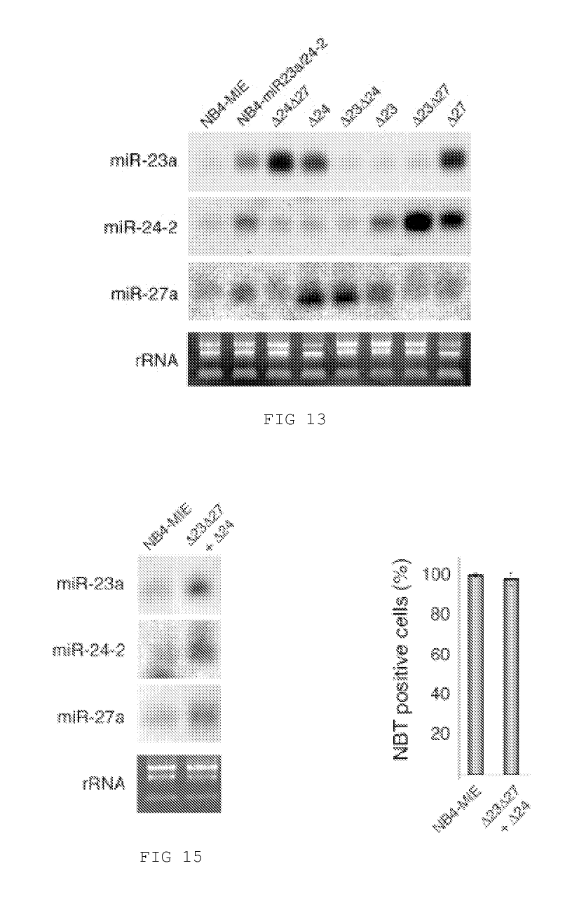

The results show that only the vector integrating the whole miR23a/24-2 precursor is able to block the differentiation of the NB4 cells in the presence of ATRA (see FIG. 12). Northern blot experiments carried out according to the protocol described above show that the vectors coding for the truncated miR23a/24-2 precursors allow for the over-expression of the miRNAs effectively coded by the latter (see FIG. 13). As a result, the co-ordinated expression of the miRNAs miR23a, miR27a and miR24-2 is necessary to obtain a blocking of the differentiation of the NB4 cells in the presence of ATRA. In order to determine whether the whole miR23a/24-2 precursor is required for the inhibition of the differentiation of the NB4 cells in the presence of ATRA, NB4 cells were co-infected by the MIE vector alone or simultaneously by the MIE-Δ23Δ27 and MIE-Δ24 vectors.

The results show that the complementation in trans of the different miRNAs of the miR23a/24-2 precursors do not block the differentiation of the NB4 cells in the presence of ATRA (see FIG. 14). However, the northern blot experiments carried out on cells infected according to the protocol described above show that the different miRNAs miR23a, miR27a and miR24-2 are over-expressed in the cells simultaneously infected by the MIE-Δ23Δ27 and MIE-Δ24 vectors (see FIG. 15). In conclusion, and unexpectedly, these experiments show that the expression of the miRNAs miR23a, miR27a and miR24-2, simultaneously and from the same precursor, has to inhibit the differentiation of the NB4 cells in the presence of ATRA.

### Example 7

### Differentiation of NB4 Cells in Response to an Inhibition of the Expression of miRNAs miR23a, miR27a and miR24-2

Chemically modified oligonucleotides (LNA®-DNA, PROLIGO) and sequences complementary to miR23a, miR27a and miR24-2 were synthesised. These modified oligonucleotides consist of nucleotide analogues containing a 2′-O, 4′-C methylene bridge that can improve both the stability of the oligonucleotide obtained as well as its hybridisation performances.

Cells from the NB4 or NB4-LR1 cell lines were then transfected by the oligonucleotides synthesised according to the protocol described in MEISTER et al. (RNA, vol. 10(3), p: 544-50, 2004). The cells of the NB4 and NB4-LR1 cell lines, transfected or not transfected by an oligonucleotide complementary to the miRNAs miR23a, miR27a and miR24-2 were then cultivated as described in example 1 in the presence or absence of ATRA or an ATRA/cAMP mixture. The evolution of the differentiation was determined on a daily basis for the different cultures as described in example 1.

In parallel, northern blot experiments were carried out according to the protocol described in example 2. The different northern blot experiments were carried out with probes complementary to the miRNAs miR23a, miR27a and miR24-2.

What is claimed:

1. A use, to manufacture a drug for the treatment of myeloid leukemia, comprised of a nucleic acid molecule chosen from among the precursor RNA miR23a/24-2 (SEQ ID NO:13), a sequence derived from such an RNA, a complementary sequence from such an RNA and a sequence derived from such a complementary sequence.

2. The use according to claim 1, wherein said drug comprises a nucleic acid molecule chosen from among a complementary sequence of the precursor RNA miR23a/24-2 (SEQ ID NO:13) and a sequence derived from such a complementary sequence.

3. The use according to claim 1, wherein said drug comprises a nucleic acid molecule presenting a sequence chosen from among:
i) the sequence of miR23a (SEQ ID NO:9), a sequence derived from miR23a, the complementary sequence of miR23a, a sequence derived from such a complementary sequence,
ii) the sequence of miR27a (SEQ ID NO:11), a sequence derived from miR27a, the complementary sequence of miR27a, a sequence derived from such a complementary sequence,
iii) the sequence of miR24-2 (SEQ ID NO:12), a sequence derived from miR24-2, the complementary sequence of miR24-2 and a sequence derived from such a complementary sequence.

4. The use according to claim 3, wherein said drug comprises a nucleic acid molecule chosen from among a complementary sequence of miR23a (SEQ ID NO:9), miR27a (SEQ ID NO:11) and miR24-2 (SEQ ID NO:12), and the sequences derived from such complementary sequences.

5. The use according to claim 1, further comprising manufacturing a drug for the treatment of a myeloid leukemia associated with a blocking of granulopoiesis.

6. The use according to claim 1, wherein the sequences of nucleic acids derived from a reference sequence present an identity of sequence of at least 80% with said sequences.

7. The use according to claim 1, wherein the length of the sequences of nucleic acids ranges from 15 to 100 nucleotides.

8. The use according to claim 1, further comprising choosing the nucleic acid molecules from among the DNA and RNA molecules.

9. The use according to claim 1, wherein the nucleic acid molecules contain one or several modified nucleotides.

10. The use according to claim 1, wherein the nucleic acid molecules are in the form of a single or double stand.

11. An in vitro method to identify effective therapeutic agents or combinations of therapeutic agents to induce the differentiation of myeloid leukemia cells, the method further comprising the stages of:
i) culturing of cells derived from a myeloid leukemia,
ii) adding at least one compound to the culture medium of said cell line,
iii) analyzing the evolution of the level of expression of at least one miRNA coded by the RNA precursor of sequence SEQ ID NO:13 between stages (i) and (ii),
iv) identifying compounds or combinations of compounds inducing a change in the level of expression of said miRNA between stages (i) and (ii).

12. The method according to claim 11, wherein stage (iii) includes the analysis of the level of expression of at least one miRNA chosen from among miR-23a (SEQ ID NO:9), miR-27a (SEQ ID NO:11) and miR-24-2 (SEQ ID NO:12).

13. The method according to claim 12, wherein stage (iv) includes the identification of the compounds or combinations of compounds modulating the level of expression of at least one miRNA chosen from among miR-23a (SEQ ID NO:9), miR-27a (SEQ ID NO:11) and miR-24-2 (SEQ ID NO:12).

14. The method according to claim 13, wherein stage (iv) includes the identification of compounds or combinations of compounds reducing the level of expression of at least one miRNA chosen from among miR-23a (SEQ ID NO:9), miR-27a (SEQ ID NO:11) and miR-24-2 (SEQ ID NO:12).

15. The method according to claim 11, wherein the compound is a therapeutic agent for the treatment of cancer.

16. The method according to claim 15, wherein the therapeutic agent is chosen from among cAMP, arsenic, the interferons, TNF, the rexinoids, retinoic acid and the retinoid derivatives.

17. The method according to claim 11, wherein stage (iii) of analysis uses the northern blot technique.

18. The method according to claim 11, wherein the cells put in culture in stage (i) are derived from a myeloid leukemia associated with a blocking of granulopoiesis.


SEQUENCE LISTING (23 sequences)

SEQ ID NO: 1 - RNA, 24 nt, Homo sapiens
caaagugcuuacagugcagguagu

SEQ ID NO: 2 - RNA, 22 nt, Homo sapiens
uaaggugcaucuagugcagaua

SEQ ID NO: 3 - RNA, 23 nt, Homo sapiens
ugugcaaaucuaugcaaaacuga

SEQ ID NO: 4 - RNA, 23 nt, Homo sapiens
ugugcaaauccaugcaaaacuga

SEQ ID NO: 5 - RNA, 22 nt, Homo sapiens
uaaagugcuuauagugcaggua

SEQ ID NO: 6 - RNA, 22 nt, Homo sapiens
uauugcacuugucccggccugu

SEQ ID NO: 7 - RNA, 980 nt, Homo sapiens
uuagaguuugagguguuaauucuaauuaucuauuucaaauuuagcaggaaaaaagagaacaucaccuuguaaaacugaagauugugaccagucagaauaaugucaaagugcuuacagugcagguagugauaugugcaucuacugcagugaaggcacuuguagcauuauggugacagcugccucgggaagccaaguugggcuuuaaagugcagggccugcugauguugagugcuuuuuguucuaaggugcaucuagugcagauagugaaguagauuagcaucuacugcccuaagugcuccuucuggcauaagaaguuauguauucauccaauaauucaagccaagcaaguauauagguguuuuaauaguuuuuguuugcaguccucuguuaguuuugcauaguugcacuacaagaagaauguaguugugcaaaucuaugcaaaacugaugguggccugcuauuuccuucaaaugaaugauuuuuacuaauuuuguguacuuuuauugugucgauguagaaucugccuggucuaucugaugugacagcuucuguagcacuaaagugcuuauagugcagguaguguuuaguuaucuacugcauuaugagcacuuaaaguacugcuagcuguagaacuccagcuucggccugucgcccaaucaaacuguccuguuacugaacacuguucuaugguuaguuuugcagguuugcauccagcugugugauauucugcugugcaaauccaugcaaaacugacugugguagugaaaagucuguagaaaaguaagggaaacucaaaccccuuucuacacagguugggaucgguugcaaugcuguguuucuguaugguauugcacuugucccggccuguugaguuugguggggauugugaccagaagauuuugaaaauuaaauauuacugaagauuucgacuuccacuguuaaauguacaagauacaugaaauauuaaagaaaauguguaa

SEQ ID NO: 8 - RNA, 20 nt, Homo sapiens
acugcagugaaggcacuugu

SEQ ID NO: 9 - RNA, 22 nt, Homo sapiens
aucacauugccagggauuucca

SEQ ID NO: 10 - DNA, 979 bp, Homo sapiens
ttagagtttgaggtgttaattctaattatctatttcaaatttagcaggaaaaaagagaacatcaccttgtaaaactgaagattgtgaccagtcagaataatgtcaaagtgcttacagtgcaggtagtgatatgtgcatctactgcagtgaaggcacttgtagcattatggtgacagctgcctcgggaagccaagttgggctttaaagtgcagggcctgctgatgttgagtgctttttgttctaaggtgatctagtgcagatagtgaagtagattagcatctactgccctaagtgctccttctggcataagaagttatgtattcatccaataattcaagccaagcaagtatataggtgttttaatagtttttgtttgcagtcctctgttagttttgcatagttgcactacaagaagaatgtagttgtgcaaatctatgcaaaactgatggtggcctgctatttccttcaaatgaatgatttttactaattttgtgtacttttattgtgtcgatgtagaatctgcctggtctatctgatgtgacagcttctgtagcactaaagtgcttatagtgcaggtagtgtttagttatctactgcattatgagcacttaaagtactgctagctgtagaactccagcttcggcctgtcgcccaatcaaactgtcctgttactgaacactgttctatggttagttttgcaggtttgcatccagctgtgtgatattctgctgtgcaaatccatgcaaaactgactgtggtagtgaaaagtctgtagaaaagtaagggaaactcaaacccctttctacacaggttgggatcggttgcaatgctgtgtttctgtatggtattgcacttgtcccggcctgttgagtttggtggggattgtgaccagaagattttgaaaattaaatattactgaagatttcgacttccactgttaaatgtacaagatacatgaaatattaaagaaaatgtgtaa

SEQ ID NO: 11 - RNA, 21 nt, Homo sapiens
uucacaguggcuaaguuccgc

SEQ ID NO: 12 - RNA, 22 nt, Homo sapiens
uggcucaguucagcaggaacag

SEQ ID NO: 13 - RNA, 950 nt, Homo sapiens
cucugccucuccaguccuggggcuggaacggagggcacagcuaggcuccagcuccccgugugguggcuccugcauaugagaaaagagcuucccugugaucaaaggaagcaucuggggaccuggaggggagguguccccaaaucucauuaccuccuuugcucucucucucuuucuccccuccaggugccagccucuggccccgcccggugccccccucaccccugugccacggccggcugggguuccuggggaugggauuugcuuccugucacaaaucacauugccagggauuuccaaccgacccugagcucugccaccgaggaugcugcccggggacgggguggcagagaggccccgaagccugugccuggccugaggagcagggcuuagcugcuugugagcaggguccacaccaagucguguucacaguggcuaaguuccgccccccaggcccucaccuccucuggccuugccgccuguccccugcugccgccugucugccugccauccugcugccuggccucccugggcucugccucccgugccuacugagcugaaacacaguugguuuguguacacuggcucaguucagcaggaacaggggucaagcccccuuggagccugcagccccugccuucccugggugggcugaugcuuggagcagagaugaggacucagaaucagaccugugucuggaggagggauguggugggugggguuggcugggcccaaaugugugcugcaggcccugauccccaacucugcaacuggggaccccugcauggccacagcucaggcugggcuguggugccagcauagauaggugggugaguggguggcccuuccauuaaaagggaagccagcuguguccuuuccgggccuggaggcuuggccccuccucucccaagccuggcaggggcacuggcccggcccgcaccuuccuagcagcc

SEQ ID NO: 14 - RNA, 22 nt, Homo sapiens
uagcagcacauaaugguuugug

SEQ ID NO: 15 - RNA, 22 nt, Homo sapiens
uagcagcacguaaauauuggcg

SEQ ID NO: 16 - RNA, 1126 nt, Homo sapiens
gaugaagaugucuuuugaaagguguacugcaaggaacaaaauguuuguaaauucuccuuuuaccaagguaaagaucaaauuuuauaaauuuacuuguuuguuuauacaaggaaaaauaacuucauauauugaauauauucaaaaguuuaagcauuuaguuguauugcccuguuaaguuggcauagcaaauaaaugcuuuucuuuuccucauuuuauucuuuguguuuccuaaccuauagcacugugcugggcacagaauggacuucaguuaaguuuuugauguagaaauguuuuauuauucuacuuaaaaucuccuuaaaaauaauuaugcauauuacaucaauguuauaauguuuaaacauagauuuuuuuacaugcauucuuuuuuuccugaaagaaaauauuuuuuauauucuuuaggcgcgaauguguguuuaaaaaaaauaaaaccuuggaguaaaguagcagcacauaaugguuuguggauuuugaaaaggugcaggccauauugugcugccucaaaaauacaaggaucugaucuucugaagaaaauauauuucuuuuuauucauagcucuuaugauagcaaugucagcagugccuuagcagcacguaaauauuggcguuaagauucuaaaauuaucuccaguauuaacugugcugcugaaguaagguugaccauacucuacaguuguguuuuaauguauauuaauguuacuaauguguuuucaguuuuauugauagucuuuucaguauuauugauaaucuuguuauuuuuaguaugauucuguaaaaaugaauuaauacuaauuuuucagauguaucaucucuuaaaauacuguaauugcaauuuaauaauuguauugaaugccaucaaguuuuuuuaaaaagcuuaugcagcauuagaggaauuuauuuuaaugcacauuuauauucaacauagacauuaauucagauuuuuacuugggauaaaacaaauucuaguuuucccuuuguuuugaaauuacuuuuaaaauaugucuuuacagauaaauauaaaauauauuaagcauuuugaacagagcuuagaagacaauauuuaguacuguuucugaauauuucuuuauaucugaaggggaaaagccauc

SEQ ID NO: 17 - RNA, 22 nt, Homo sapiens
ugagguaguagguuguauaguu

SEQ ID NO: 18 - RNA, 21 nt, Homo sapiens
agagguaguagguugcauagu

SEQ ID NO: 19 - RNA, 22 nt, Homo sapiens
uagcagcacaucaugguuuaca

SEQ ID NO: 20 - RNA, 20 nt, Homo sapiens
cauaaaguagaaagcacuac

SEQ ID NO: 21 - RNA, 21 nt, Homo sapiens
ugucaguuugucaaauacccc

SEQ ID NO: 22 - RNA, 23 nt, Homo sapiens
aaaagcuggguugagagggcgaa

SEQ ID NO: 23 - RNA, 22 nt, Homo sapiens
cuggacuuggagucagaaggcc